(19) United States
(12) Patent Application Publication
(10) Pub. No.: US 2023/0293119 A1
(43) Pub. Date: Sep. 21, 2023

(54) TELEHEALTH IMAGING AND ROBOTICS

(71) Applicant: Welch Allyn, Inc., Skaneateles Falls, NY (US)

(72) Inventors: Gene J. Wolfe, Pittsford, NY (US); Patrice Etchison, Cary, NC (US); Craig M. Meyerson, Syracuse, NY (US); Daniel Shirley, Raleigh, NC (US); Carlos Andres Suarez, Syracuse, NY (US)

(21) Appl. No.: 18/178,815

(22) Filed: Mar. 6, 2023

Related U.S. Application Data
(60) Provisional application No. 63/269,424, filed on Mar. 16, 2022.

(51) Int. Cl. A61B 5/00; G16H 80/00; A61B 34/35; G06V 40/16; G06T 7/00; H04N 7/18

(52) U.S. Cl. CPC A61B 5/7465; G16H 80/00; A61B 5/4836; A61B 5/0077; A61B 5/445; A61B 34/35; G06V 40/161; G06T 7/0014; H04N 7/183; A61B 2576/00; H04N 23/695

(57) ABSTRACT

A method of remote monitoring a patient includes monitoring the patient under a first modality that blocks identification of the patient. The method includes determining whether an event is detected by the first modality. When an event is detected, the method includes monitoring the patient under a second modality, which is different from the first modality and includes capturing images of the patient.

### BACKGROUND

Telehealth systems often require the ability to appropriately view a patient to assess their health condition. However, the patient and their environment are not often easily visualized by traditional camera equipment. For example, close-up details of the patient can be difficult for a remote clinician to view and interpret through traditional camera equipment during a telehealth consultation. Additionally, security of health data is becoming increasingly important in view of the growing prevalence of telehealth consultations between patients and remote clinicians.

### SUMMARY

In general terms, the present disclosure relates to telehealth. In one possible configuration, a system provides improved imaging and diagnostic analysis during a telehealth consultation, while also improving security of protected health information. Various aspects are described in this disclosure, which include, but are not limited to, the following aspects.

One aspect relates to a method of remote monitoring a patient, the method comprising: monitoring the patient under a first modality, wherein the first modality blocks identification of the patient; determining whether an event is detected by the first modality; and when an event is detected, monitoring the patient under a second modality, wherein the second modality includes capturing images of the patient.

Another aspect relates to a method of conducting a telehealth consultation, the method comprising: receiving a request from a patient to start a telehealth consultation; obtaining a live image of the patient; obtaining a stored image of the patient; comparing the live image of the patient with the stored image of the patient; and when the live image of the patient matches the stored image of the patient, initiating the telehealth consultation with a clinician remotely located with respect to the patient.

Another aspect relates to a method of conducting a telehealth consultation, the method comprising: receiving images of a patient; receiving one or more physiological parameters of the patient; performing a diagnostic analysis on the patient based on at least one of the images and the one or more physiological parameters; and instructing a robotic arm to perform a procedure based on the diagnostic analysis.

Another aspect relates to a telehealth device, comprising: at least one processing device; and a memory device storing instructions which, when executed by the at least one processing device, cause the telehealth device to: control operation of a diagnostic imager for capturing images of a patient during a telehealth consultation; analyze the images of the patient to determine a disease state; and provide a clinical recommendation based on the disease state.

### DESCRIPTION OF THE FIGURES

The following drawing figures, which form a part of this application, are illustrative of the described technology and are not meant to limit the scope of the disclosure in any manner.

FIG. 1 schematically illustrates an example of a telehealth system.

FIG. 2 schematically illustrates an example of a method of remote monitoring a patient using the telehealth system of FIG. 1.

FIG. 3 schematically illustrates an example of a monitoring device of the telehealth system of FIG. 1.

FIG. 4 schematically illustrates an example of a method of conducting a telehealth consultation with a patient using the telehealth system of FIG. 1.

FIG. 5 schematically illustrates an example of a first embodiment of a diagnostic imager of the telehealth system of FIG. 1.

FIG. 6 is an isometric view of the diagnostic imager of FIG. 5.

FIG. 7 schematically illustrates an example of a second embodiment of the diagnostic imager of the telehealth system of FIG. 1.

FIG. 8 is an isometric view of the diagnostic imager of FIG. 7.

FIG. 9 schematically illustrates an example of a clinical decision support tool that uses data acquired from the diagnostic imagers of FIGS. 5-8.

FIG. 10 schematically illustrates an example of a robotic arm that is included in some examples as part of the telehealth system of FIG. 1.

FIG. 11 is an isometric view of the robotic arm of FIG. 10.

FIG. 12 schematically illustrates an example of a method of conducting a telehealth consultation with a patient using the robotic arm of FIGS. 10 and 11.

### DETAILED DESCRIPTION

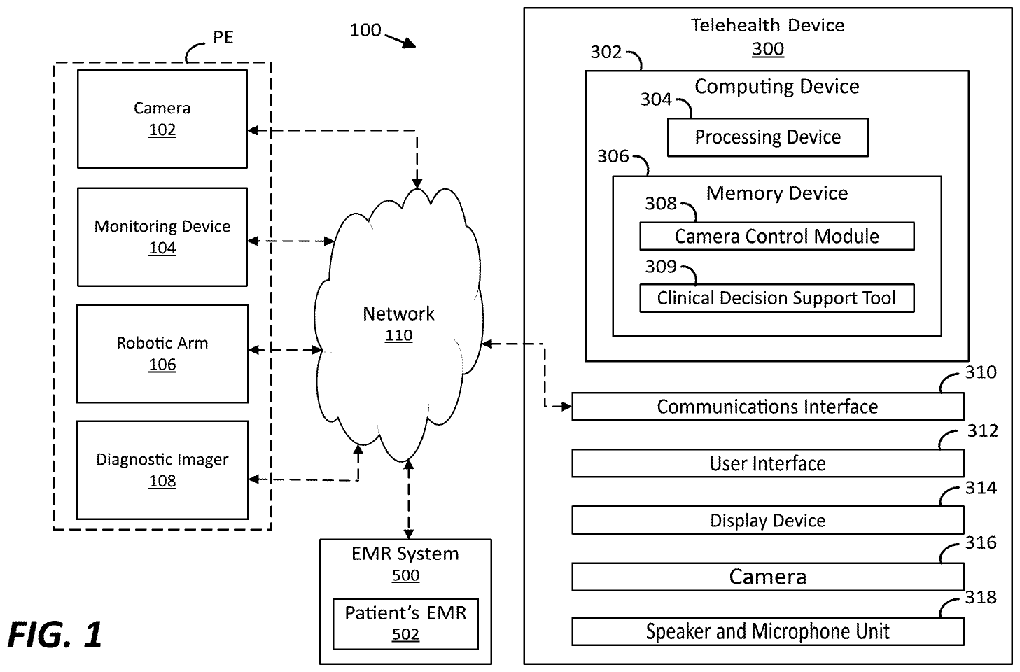

FIG. 1 schematically illustrates an example of a telehealth system 100. As described herein, telehealth is the distribution of health-related services and information via electronic information and telecommunication technologies.

In the example shown in FIG. 1, the telehealth system 100 includes a telehealth device 300 that is in communication with various devices located in a patient environment PE via a communications network 110. For example, the telehealth device 300 is in communication via the communications network 110 with a camera 102, a monitoring device 104, a robotic arm 106, and a diagnostic imager 108 each located in the patient environment PE.

The telehealth device 300 is remotely located with respect to the patient environment PE. In one example, the patient environment PE is a patient room within a healthcare facility such as a hospital, a nursing home, a long-term care facility, and the like. In such examples, the telehealth device 300 can be located in a different location within the healthcare facility such as in a nurses' station or in a control room. In further examples, the telehealth device 300 can be located offsite such as in a separate building, campus, or other remote geographical location.

In another example, the patient environment PE is a patient's home. In such examples, the telehealth device 300 can be located in a healthcare facility such as a hospital such that the telehealth device 300 can be used to provide hospital-at-home healthcare services.

The communications network 110 can include any type of wired or wireless connections or any combinations thereof. The communications network 110 includes the Internet. In some examples, the communications network 110 includes wireless connections such as cellular network connections including 4G or 5G. Wireless connections can also be accomplished using Wi-Fi, ultra-wideband (UWB), Bluetooth, and the like.

The telehealth device 300 is also in communication with an electronic medical record (EMR) system 500 via the communications network 110. The telehealth device 300 can acquire health information from an electronic medical record (EMR) 502 (alternatively termed electronic health record (EHR)) of a patient that is stored in the EMR system 500. The EMR 502 of the patient includes health information such as lab results, scans, administered medications, health interventions including surgeries and procedures performed on the patient, one or more images of the patient taken by the camera 102 and/or diagnostic imager 108, records of the physiological parameters acquired from the monitoring device 104, and other health information.

The telehealth device 300 includes a computing device 302 having a processing device 304 and a memory device 306. The processing device 304 is an example of a processing unit such as a central processing unit (CPU). The processing device 304 can include one or more CPUs. In some examples, the processing device 304 can include one or more microcontrollers, digital signal processors, field-programmable gate arrays, or other electronic circuits.

The memory device 306 operates to store data and instructions for execution by the processing device 304. The memory device 306 includes computer-readable media, which may include any media that can be accessed by the telehealth device 300. The computer-readable media can include computer readable storage media and computer readable communication media. As shown in FIG. 1, the memory device 306 stores at least one of a camera control module 308 to control operation of the camera 102, and a clinical decision support tool 309 that analyzes data from the diagnostic imager 108 to provide clinical decision support during a telehealth consultation conducted using the telehealth device 300. The camera control module 308 and the clinical decision support tool 309 will each be described in more detail below.

Computer readable storage media includes volatile and nonvolatile, removable, and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the telehealth device 300. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The telehealth device 300 includes a communications interface 310 that operates to connect the telehealth device 300 to the communications network 110 for communication with the devices in the patient environment PE such as the camera 102, the monitoring device 104, the robotic arm 106, and the diagnostic imager 108, and for communication with the EMR system 500. The communications interface 310 can include both wired interfaces (e.g., USB ports, and the like) and wireless interfaces (e.g., Bluetooth, Wi-Fi, and similar types of protocols).

The telehealth device 300 further includes a user interface 312 for accepting inputs from a remote clinician who uses the telehealth device 300. The inputs received from the user interface 312 can be used to control one or more devices in the patient environment PE including the camera 102, the monitoring device 104, the robotic arm 106, and the diagnostic imager 108.

The telehealth device 300 further includes a display device 314 for displaying image and/or video data of the patient captured by the camera 102, physiological variables measured by the monitoring device 104, and images captured by the diagnostic imager 108. In some examples, the display device 314 is a touchscreen such that it can also function as a user interface that receives inputs for controlling one or more devices in the patient environment PE.

The telehealth device 300 further includes a camera 316 for capturing a video stream of a remote clinician who uses the telehealth device 300, and a speaker and microphone unit 318 for outputting audio of the patient captured in the patient environment PE, and for capturing audio of the remote clinician for playback in the patient environment PE. In view of the foregoing, the camera 316 and the speaker and microphone unit 318 enable the telehealth system 100 to provide two-way video communications between the remote clinician who uses the telehealth device 300 and a patient or a local caregiver located in the patient environment PE.

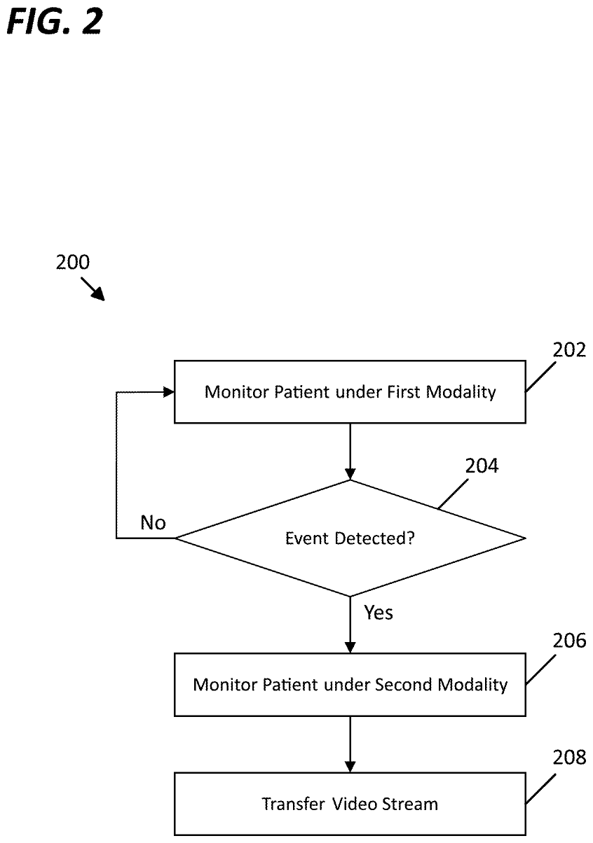

FIG. 2 schematically illustrates an example of a method 200 of remote monitoring a patient located in the patient environment PE using the telehealth system 100. In some examples, the method 200 is performed by the camera control module 308 stored on the memory device 306 of the telehealth device 300. As shown in FIG. 2, the method 200 includes an operation 202 of monitoring the patient in the patient environment PE under a first modality.

In some instances, data under the first modality is captured by the camera 102. As an illustrative example, the camera 102 can be mounted to a fixture inside the patient environment such as a wall or ceiling, can be mounted on furniture inside the patient environment PE, or can be mounted on another device in the patient environment PE such as the monitoring device 104, a patient support system such as a hospital bed, or other devices.

The first modality captures data of the patient in the patient environment PE that does not uniquely identify the patient. For example, the first modality can include capturing images of the patient under a first spectrum of light that obfuscates the patient such that the patient is not uniquely identifiable from the data captured under the first modality. As an illustrative example, the first spectrum of light can include infrared and/or far infrared spectrums. In another example, the first spectrum of light can include visible light having a low resolution such that it is not possible to identify the patient from low resolution images that are generated. The data captured under the first modality can be used to monitor a location or a status of the patient in the patient environment PE, such as whether the patient remains in bed, whether the patient exits the bed, whether the patient has absconded the patient environment PE, or whether the patient has experienced a fall in the patient environment PE, while preventing identification of the patient.

By removing data that can be used to identify the patient, the data captured under the first modality does not contain protected health information (PHI), also referred to as personal health information, which is defined by the Health Insurance Portability and Accountability Act (HIPAA) as any data related to past, present or future health of an individual, the provision of healthcare to the individual, or the payment for the provision of healthcare to the individual. PHI can include demographic information, medical histories, test and laboratory results, mental health conditions, insurance information, and other data. HIPAA regulates how PHI data is created, collected, transmitted, maintained, and stored by a covered organization.

In one example, the first modality includes using light detection and ranging (lidar) technology to detect the location and status of the patient inside the patient environment PE. Lidar is a method for determining variable distances by targeting an object with a laser and measuring the time for the reflected light to return to the receiver. In such examples, the camera 102 can be equipped with a lidar sensor to capture lidar data under the first modality.

In another example, the first modality includes transmitting millimeter waves (sometimes abbreviated MMW or mmWave) to detect the location and status of the patient inside the patient environment PE. Millimeter waves are electromagnetic (i.e., radio) waves that are typically within the frequency range of 30-300 gigahertz (GHz). In such examples, the camera 102 can be equipped with an antenna to transmit and receive millimeter waves for capturing the data of the patient in the patient environment under the first modality. In some instances, the camera 102 and/or the monitoring device 104 include aspects of the patient monitoring systems described in U.S. Pat. No. 10,548,476, issued on Feb. 4, 2020, and U.S. patent application Ser. No. 16/748,293, filed on Jan. 21, 2020, which are incorporated herein by reference in their entireties.

The lidar and millimeter wave data in the examples described above reduces bandwidth requirements for communicating and storing the patient data captured under the first modality. For example, the lidar and millimeter wave data can reduce bandwidth requirements for communicating the data from the patient environment PE to a remote location such as where the telehealth device 300 is located, and for storing the data in the patient's EMR 502.

In further examples, the first modality can include capturing a red, green, blue-depth (RGB-D) video feed that augments a conventional video image with depth information. In such examples, the RGB-D video feed includes data representative of a distance of an object (e.g., the patient in the patient environment PE) in a per-pixel basis. The RGB-D video feed can obscure the patient's face and objects in the patient environment PE that can identify the patient.

In further examples, the first modality can include capturing video images of the patient having a low resolution. In such examples, the low resolution obfuscates or blurs the patient's face and body such that the patient cannot be uniquely identified from the video images.

Next, the method 200 includes an operation 204 of determining whether an event is detected by the first modality. An event can include conditions and hazards that can potentially cause harm to the patient. For example, an event can include a patient fall or patient abscondence (i.e., the patient leaves the patient environment PE without authorization).

When no event is detected (i.e., “No” at operation 204), the method 200 returns to operation 202 and continues to monitor the patient under the first modality. When an event is detected (i.e., “Yes” at operation 204), the method 200 proceeds to an operation 206 of monitoring the patient in the patient environment PE under a second modality.

The second modality can include capturing data of the patient in the patient environment PE under a second spectrum of light that does not obfuscate the patient. In such examples, the patient is identifiable from the images captured under the second modality. The second spectrum can include the visible spectrum of light. In examples where the first modality includes capturing images in the visible spectrum of light having a low resolution, the second modality can include capturing the images with a higher resolution such that additional details not identifiable in the first modality can be seen. The images captured under the second modality with a higher resolution can aid assessment of the patient by a remote clinician.

In view of the foregoing, the method 200 enables the telehealth system 100 to capture data containing PHI of the patient for transmission and display only when such data is clinically necessary for direct observations and medical interventions. Otherwise, the patient is monitored under first modality which obscures the patient's identity and does not include PHI.

The method 200 enables the telehealth system 100 to monitor the patient for long running continuous periods of time under the first modality, which can bolster PHI data security by not recording or transmitting visible video data of the patient that can be used to identify the patient, while also reducing bandwidth requirements. Additionally, the method 200 enables the telehealth system 100 to record, transmit and display high resolution video data when clinically necessary for direct observations and medical interventions. The method 200 can reduce data leak concerns for PHI by reducing exposure on which a malicious actor can operate.

In some examples, the method 200 includes an operation 208 of transferring the transmission of the video data under the second modality to another device operated by a clinician who has permission to view the PHI. In some examples, operation 208 is performed prior to operation 206 such that an operator in a first remote location receives data captured under the first modality for monitoring the patient, but does not receive video data of the patient containing PHI. Instead, only an authorized clinician in a second remote location is able to view the video data containing PHI. This can further reduce PHI data leak exposure.

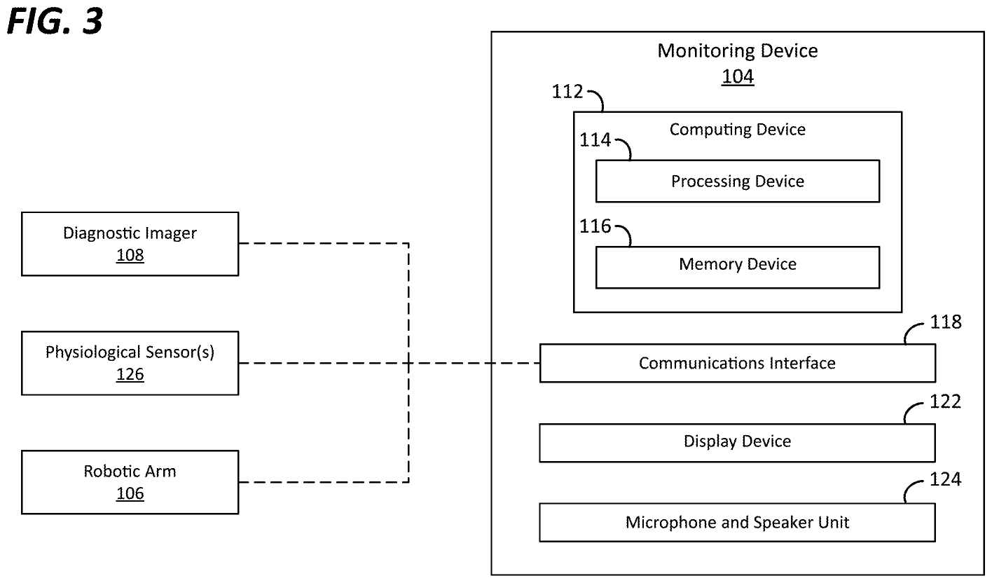

FIG. 3 schematically illustrates an example of the monitoring device 104. In some examples, the monitoring device 104 is a spot monitor, similar to the one described in U.S. Pat. No. 9,265,429, which is herein incorporated by reference in its entirety.

In the example illustrated in FIG. 3, the monitoring device 104 includes a computing device 112 having a processing device 114 and a memory device 116 that can be similar to the computing device 302, the processing device 304, and the memory device 306 of the telehealth device 300 described above. The monitoring device 104 further includes a communications interface 118 for connection to one or more physiological sensors 126 for measuring and recording physiological parameters of the patient in the patient environment PE. The communications interface 118 can include both wired interfaces (e.g., USB ports, and other types of ports) and wireless interfaces (e.g., Bluetooth, Wi-Fi, and other types of wireless protocols). Examples of the physiological sensors 126 can include sensors for measuring and recording blood oxygen saturation (SpO2), non-invasive blood pressure (systolic and diastolic), respiration rate, pulse rate, temperature, electrocardiogram (ECG), heart rate variability, and the like.

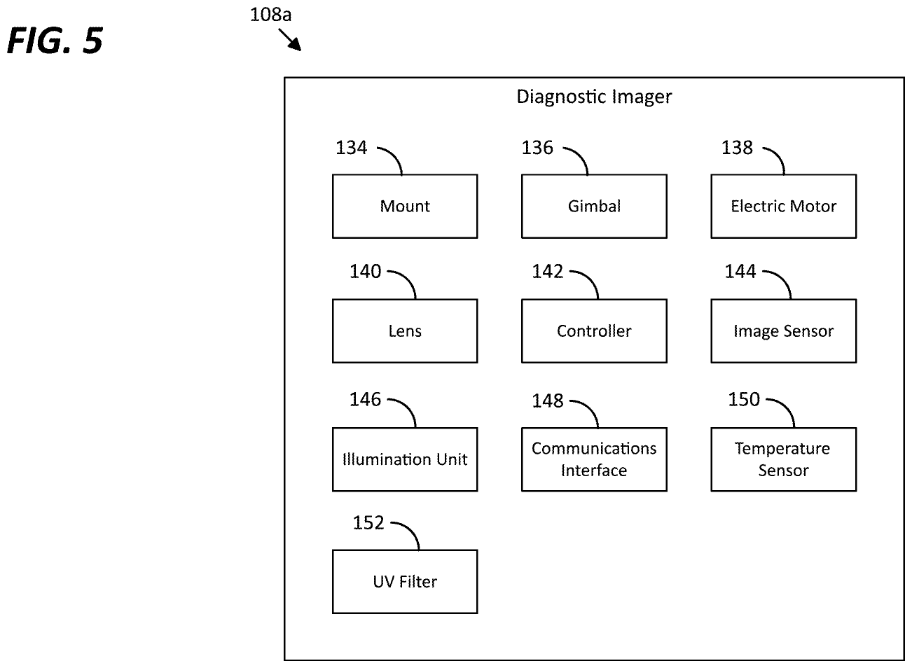

Additionally, the diagnostic imager 108 can connect to the monitoring device 104 via the communications interface 118 to send images of the patient to the monitoring device 104 for analysis and display on a display device 122 of the monitoring device. Illustrative examples of the diagnostic imager 108 will be described in more detail with reference to FIGS. 5-8.

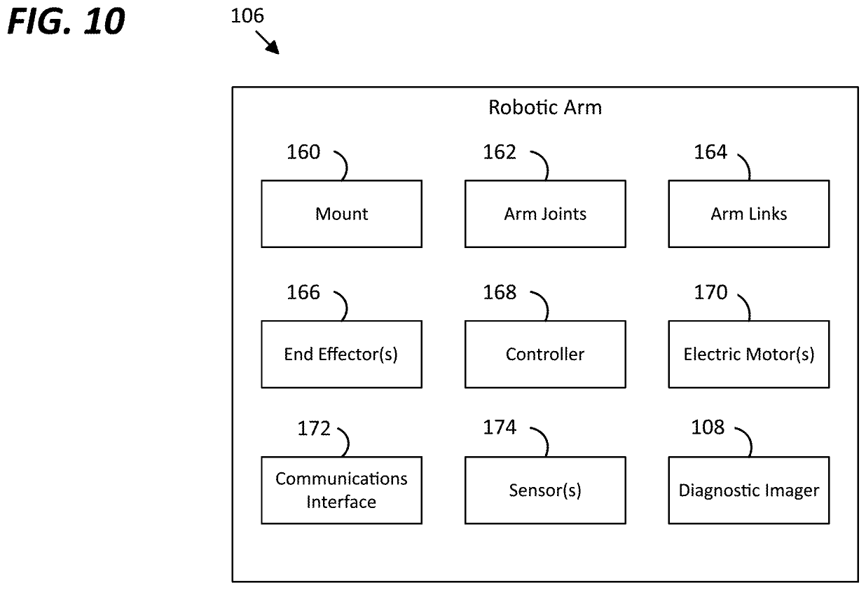

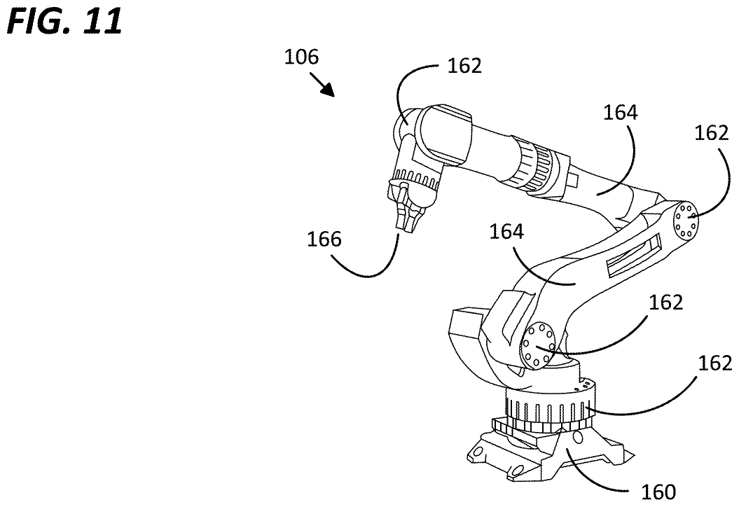

In the example shown in FIG. 3, the robotic arm 106 connects to the communications interface 118 of the monitoring device 104 through wired, wireless, or any combination of wired and wireless connections. In alternative examples, the robotic arm 106 does not connect to the monitoring device 104, and is separately controlled by the telehealth device 300. Examples of the robotic arm 106 will be described in more detail with reference to FIGS. 10 and 11.

In the example shown in FIG. 3, the monitoring device 104 includes a display device 122 and a microphone and speaker unit 124. The display device 122 and the microphone and speaker unit 124 can enable two-way communications between a patient or local caregiver in the patient environment PE and a clinician operating the telehealth device 300 in a remote location. For example, a video feed of the clinician captured by the camera 316 of the telehealth device 300 is displayable on the display device 122 of the monitoring device 104. Also, audio of the clinician captured by the speaker and microphone unit 318 of the telehealth device 300 is outputted on the microphone and speaker unit 124 of the monitoring device 104.

Additionally, the microphone and speaker unit 124 of the monitoring device 104 can capture audio of the patient or local caregiver in the patient environment PE for output on the speaker and microphone unit 318 of the telehealth device 300. Also, the camera 102 or the diagnostic imager 108 can capture video data of the patient or a local caregiver in the patient environment PE for display on the display device 314 of the telehealth device 300.

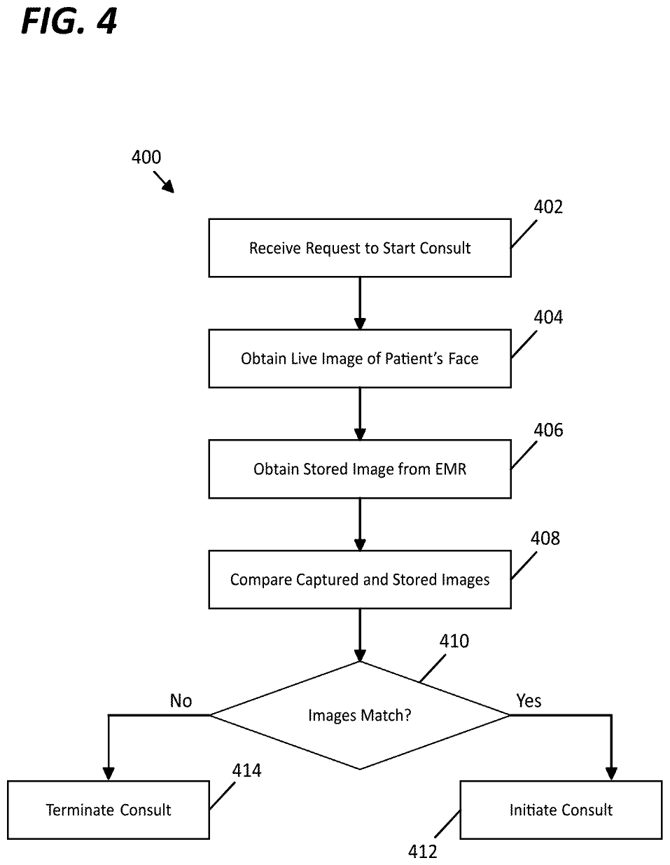

FIG. 4 schematically illustrates an example of a method 400 of conducting a telehealth consultation with a patient using the telehealth system 100. In some examples, the method 400 can be performed by the telehealth device 300. As will be described in more detail, the method 400 can provide enhanced security and protect against healthcare fraud.

As shown in FIG. 4, the method 400 includes an operation 402 of receiving a request to start a telehealth consultation. The request to start the telehealth consultation can be received from a device operated by the patient in the patient environment PE such as a smartphone, tablet computer, laptop, or other computing device. In some examples, the request is received by the telehealth device 300 from the patient's device via the communications network 110.

Next, the method 400 includes an operation 404 of obtaining a live image of the patient's face. In some examples, the live image of the patient's face is obtained by the telehealth device 300 through a camera integrated with or otherwise connected to the device operated by the patient in the patient environment PE. In further examples, the live image of the patient's face is obtained by the telehealth device 300 via the camera 102 inside the patient environment.

Next, the method 400 includes an operation 406 of obtaining a stored image of the patient's face. The stored image can be an image that was captured during a previous telehealth consultation, or that was captured when the patient was admitted to a healthcare facility such as hospital. In some examples, the stored image is obtained by the telehealth device 300 from the patient's electronic medical record (EMR) 502 via the communications network 110.

Next, the method 400 includes an operation 408 of comparing the live image of the patient obtained in operation 404 with the stored image of the patient obtained in operation 406. The comparison can use facial recognition technology to extract certain facial features for comparing the live image of the patient with the stored image of the patient.

Next, the method 400 includes an operation 410 of determining whether the live image of the patient captured in operation 404 matches the stored image of the patient obtained in operation 406 to validate or verify the identity of the patient. When the image of the patient captured in operation 404 matches with the image of the patient obtained in operation 406 (i.e., “Yes” in operation 410), the method 400 proceeds to operation 412 of initiating the telehealth consultation with a clinician remotely located with respect to the patient environment (i.e., a clinician who is operating the telehealth device 300 shown in FIG. 1).

Otherwise, when the image of the patient captured in operation 404 does not match the image of the patient obtained in operation 406 (i.e., “No” in operation 410), the method 400 proceeds to operation 414 of terminating the telehealth consultation because the identity of the person requesting the telehealth consultation is not verified. In view of the foregoing, the method 400 protects against healthcare fraud and protects protected health information (PHI) by blocking impostors who pretend to be a patient from initiating a telehealth consultation.

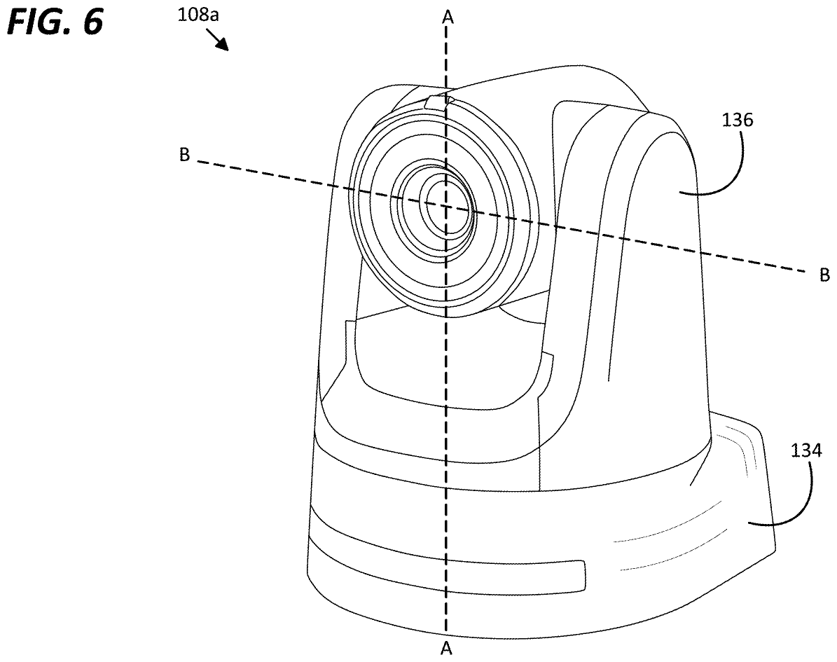

FIG. 5 schematically illustrates an example of a first embodiment of the diagnostic imager 108a. FIG. 6 is an isometric view of the diagnostic imager 108a. The diagnostic imager 108a is configured to capture high quality images of a patient for analyzing close up details of the patient such as conditions on their face and extremities, and to more clearly and accurately image the patient environment PE for assessment by a remote clinician operating the telehealth device 300 to improve patient diagnosis, well-being, and plan of care. The diagnostic imager 108a is configured to capture well illuminated and high-resolution images that can be directed to any position for enhancing an assessment of the patient by the remote clinician.

Referring now to FIGS. 5 and 6, the diagnostic imager 108a includes a mount 134 that operates to fix the diagnostic imager 108a to a surface. In one example, the mount 134 fixes the diagnostic imager 108a to the monitoring device 104. In further examples, the mount 134 fixes the diagnostic imager 108a to a fixture such as a wall or ceiling in the patient environment PE, or to a piece of furniture or another device in the patient environment PE.

The diagnostic imager 108a further includes a gimbal 136 that operates to provide pivoted support for the diagnostic imager 108a. The pivoted support permits rotation of the diagnostic imager 108a to adjust a field of view of the diagnostic imager 108a. For example, the gimbal 136 allows the diagnostic imager 108a to pan left and right about a first axis A-A, and to tilt up and down about a second axis B-B to adjust a field of view of the diagnostic imager 108a. In such examples, the diagnostic imager 108a is a pan-tilt-zoom (PTZ) camera.

The diagnostic imager 108a further includes an electric motor 138 that operates to move the gimbal 136 to pan left and right about the first axis A-A, and to move the gimbal 136 to tilt up and down about the second axis B-B. The electric motor 138 receives commands from a controller 142 to control the pan and tilt of the diagnostic imager 108a.

In some examples, the electric motor 138 also operates to move a lens 140 to control an optical zoom of the diagnostic imager 108a. For example, the lens 140 is mounted in front of an image sensor 144, and the electric motor 138 is operable to move the lens 140 relative to the image sensor 144 to adjust a focal length of the diagnostic imager 108a to zoom-in and zoom-out. The electric motor 138 can operate to move the lens 140 based the commands received from the controller 142. Alternatively, the controller 142 can perform digital zoom on the images captured by the image sensor 144 such as by enlarging pixels within an area of interest.

The controller 142 is an example of a computing device that can control operation of the diagnostic imager 108a. For example, the controller 142 can instruct the electric motor 138 to move the gimbal 136 such as to pan the image sensor 144 left and right, and to tilt the image sensor 144 up and down. Additionally, the controller 142 can instruct the electric motor 138 to move the lens 140 relative to the image sensor 144 to adjust the optical zoom. The lens 140 and image sensor 144 can be used to capture high resolution images of an area of interest.

The diagnostic imager 108a further includes a communications interface 148 that operates to provide communications between the diagnostic imager 108a and other devices such as the monitoring device 104. For example, the communications interface 148 can include a wired or wireless connection to the communications interface 118 of the monitoring device 104. In further examples, the communications interface 148 can include a wired or wireless connection to the communications network 110 for communications with the telehealth device 300. The communications interface 148 can include both wired interfaces (e.g., USB ports, and the like) and wireless interfaces (e.g., Bluetooth, Wi-Fi, and other types of protocols).

In some examples, the communications interface 148 receives commands from the telehealth device 300 to adjust the pan, tilt, and zoom of the diagnostic imager 108a for imaging an object, surface area of interest, or anatomical position during a telehealth consultation. In further examples, the controller 142 stores algorithms that automatically adjust the pan, tilt, and zoom of the diagnostic imager 108a for imaging a designated object, surface area of interest, or anatomical position. In some examples, the algorithms program the diagnostic imager 108a to image or scan an activity in the patient environment PE during predetermined intervals of time. In further examples, the controller 142 receives commands via a user interface on the monitoring device 104 (e.g., on the display device 122 in examples where it is a touchscreen display) or the diagnostic imager 108a itself to locally control the pan, tilt, and zoom of the diagnostic imager 108a for imaging an object, surface area of interest, or anatomical position.

As further shown in FIG. 5, the diagnostic imager 108a includes an illumination unit 146 that operates to illuminate a surface for taking images using the lens 140 and the image sensor 144. The illumination unit 146 is configured to improve the fidelity, quality, and resolution of images captured by the diagnostic imager 108a for display on the display device 314 of the telehealth device 300 during a telehealth consultation with a remote clinician. For example, the illumination unit 146 is configured to more accurately depict facial droop, pupil size, skin color, and other visual features for display on the display device 314 of the telehealth device 300 to improve remote clinician assessment during the telehealth consultation.

The illumination unit 146 can emit visible light and infrared light for imaging the patient. In some instances, the illumination unit 146 can also emit ultraviolet light.

The illumination unit 146 is controllable by the controller 142 to provide precise illumination, and to maintain uniformity and consistency for imaging a surface such as the patient's face, or a wound, abrasion, laceration, and the like. The illumination unit 146 is further configured to prevent single bright flashes which can blanch fine details of a surface such as skin, and which can wash out color and tone on areas of interest such as wounds and markings.

The illumination unit 146 is controllable to adjust diffusion, angulation, and intensity of lighting based on the task being performed by the diagnostic imager 108a and/or the object, surface, or area of interest being imaged. As an illustrative example, the illumination unit 146 can include a ring of light-emitting diodes (LEDs) that are positioned around a periphery of the diagnostic imager 108 such as around the lens 140 and/or image sensor 144.

When uniform lighting is desirable, the LEDs are uniformly illuminated around the periphery of the diagnostic imager 108. When it is desirable to have a desired angulation (e.g., for measuring a depth of a wound via penumbra effect), the angulation of the illumination unit 146 is adjustable by illuminating only a subset of the LEDs around the periphery of the diagnostic imager 108. For example, the angulation can be adjusted by illuminating a subset of LEDs on an upper portion; illuminating a subset of LEDs on a lower portion; illuminating a subset of LEDs on a left side; and illuminating a subset of LEDs on a right side. Additional examples for changing the angulation of the illumination unit 146 are contemplated.

Additionally, the illumination unit 146 is controllable to adjust color temperature, which is the light appearance provided by the illumination unit 146 measured in degrees of Kelvin (K). In some examples, the diagnostic imager 108a or the monitoring device 104 can communicate with other devices in the patient environment PE to adjust ambient light to further enhance the illumination of the imaging provided by the diagnostic imager 108a.

In some examples, a clinician remotely located with respect to the patient environment PE can enter one or more inputs into the telehealth device 300 to control the diffusion, angulation, intensity, color temperature, and ambient light parameters during a telehealth consultation with the patient in the patient environment PE. In further examples, the controller 142 of the diagnostic imager 108a stores algorithms that optimize the diffusion, angulation, intensity, color temperature, and ambient light parameters during a telehealth consultation between the remote clinician and the patient in the patient environment PE.

Additionally, the controller 142 stores algorithms for moving the diagnostic imager 108a to scan close surfaces (e.g., skin wounds), monitor activities in the patient environment PE (e.g., patient motion), and/or monitor conditions inside the patient environment PE itself for accurate visualization and analysis. For example, the controller 142 can be programmed to recognize frontal, oblique, lateral, and other anatomical views of the patient via facial recognition. These algorithms are part of the clinical decision support tool 309 that uses data acquired from the diagnostic imager 108a, which will be described in more detail.

As further shown in FIG. 5, the diagnostic imager 108a includes a temperature sensor 150 that operates to measure temperature readings of a surface being imaged by the diagnostic imager 108a. The temperature sensor 150 measures the temperature readings without contacting the surface such that it is a non-contact or contactless temperature sensor.

In some examples, the temperature sensor 150 is an infrared thermometer. For example, the temperature sensor 150 may include an infrared temperature sensor such as a thermopile or similar infrared-based temperature sensing devices. The temperature readings measured by the temperature sensor 150 can be used by the diagnostic imager 108a or the monitoring device 104 for detecting fever, infection, and other disease states.

As further shown in FIG. 5, the diagnostic imager 108a includes an ultraviolet (UV) light filter 152 operable to remove UV light from the images captured by the image sensor 144. This can further enhance the lighting characteristics of the images captured by the diagnostic imager 108a for more accurate analysis of an area of interest and assessment of the patient.

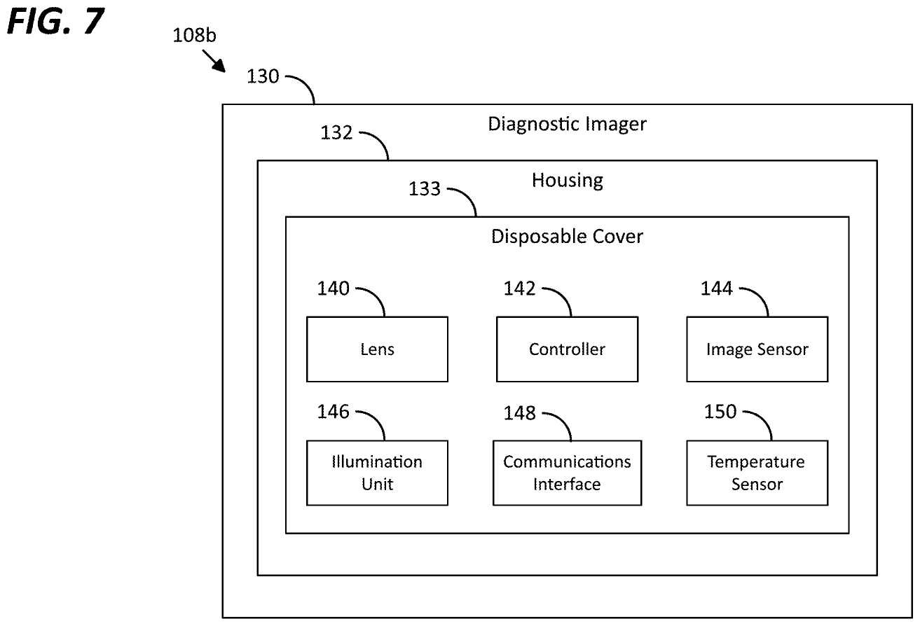

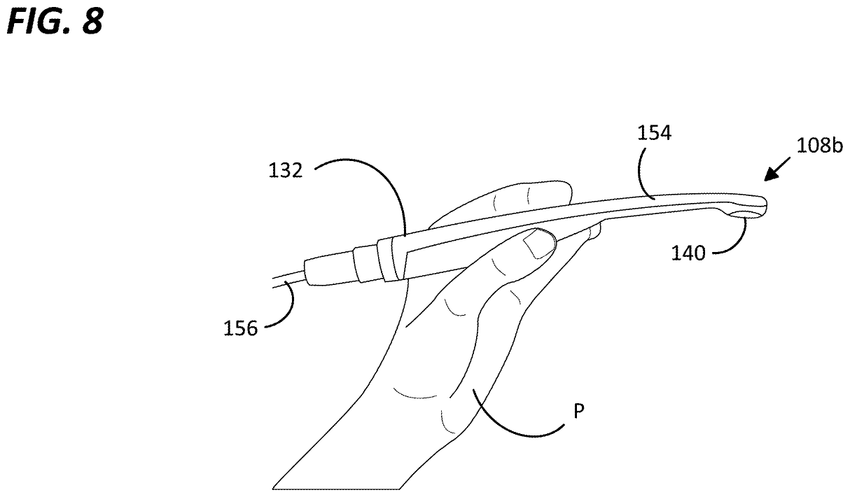

FIG. 7 schematically illustrates an example of a second embodiment of the diagnostic imager 108b. FIG. 8 is an isometric view of the diagnostic imager 108b being held by a patient P. In this embodiment, the diagnostic imager 108b is a handheld portable imager. The diagnostic imager 108b includes the lens 140, the controller 142, the image sensor 144, the illumination unit 146, the communications interface 148, and the temperature sensor 150, previously described.

As shown in FIG. 8, the diagnostic imager 108b includes a housing 132 that is shaped and sized for handheld use. The housing 132 has an elongated neck 154 that resembles a selfie-stick. The lens 140, the image sensor 144, the illumination unit 146, and the temperature sensor 150 can each be positioned toward the distal end of the elongated neck 154. This enables the patient P to position and orientate the diagnostic imager 108b to image hard to reach areas such as the patient's back and other posterior areas. In some examples, the length of the elongated neck 154 is adjustable. In further examples, the elongated neck 154 is rotatable. The adjustable length and/or rotation of the elongated neck 154 allow the patient P to optimize the length and orientation of the diagnostic imager 108b for imaging hard to reach areas on the patient's body.

The housing 132 can be made of a durable material that allows the diagnostic imager 108b to be cleaned and sanitized for re-use in multiple patient environments. In some examples, the housing 132 includes a disposable cover 133 that can be discarded after each use of the diagnostic imager 108b to sanitize the diagnostic imager 108b for re-use.

The lens 140 includes a macro lens for extreme close-up imaging of an area of interest on the body of the patient. Also, the image sensor 144 is configured for high resolution imaging. The illumination unit 146 is configured for adjustable illumination. For example, the diffusion, angulation, intensity, and color temperature of the illumination unit 146 are adjustable.

In FIG. 8, the diagnostic imager 108b includes a cable 156 that can plug into the communications interface 118 of the monitoring device 104 for providing two-communications between the diagnostic imager 108b and the monitoring device 104. In further examples, the diagnostic imager 108b can wirelessly connect to the communications interface 118 of the monitoring device 104 such that the diagnostic imager 108b is cordless.

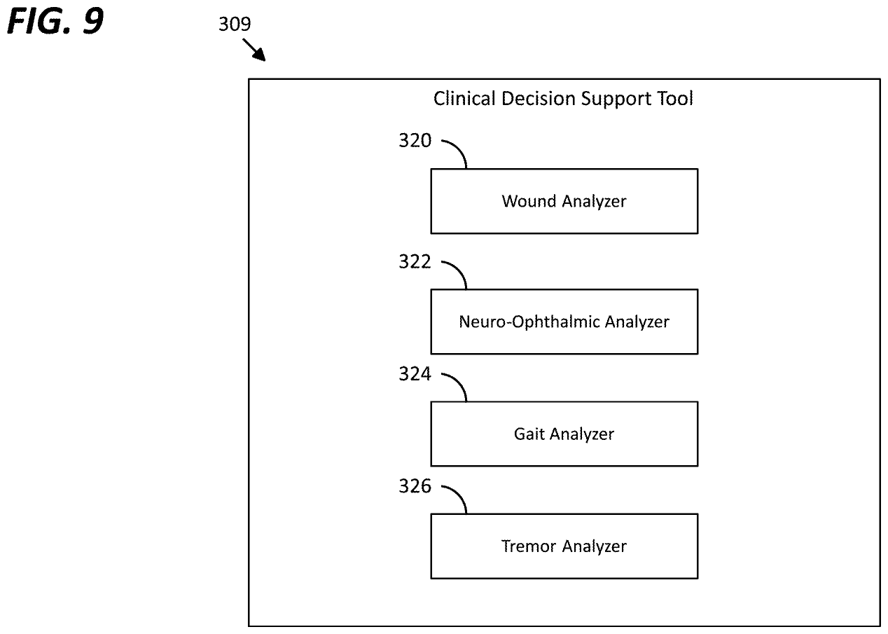

FIG. 9 schematically illustrates an example of the clinical decision support tool 309 that uses data acquired from the diagnostic imagers 108a, 108b to improve assessment of a patient in the patient environment PE during a telehealth consultation. The clinical decision support tool 309 can detect and classify warning signs of the patient while the patient is engaged in the telehealth consultation with a remote clinician. As described above, the clinical decision support tool 309 can be installed on the memory device 306 of the telehealth device 300 (see FIG. 1). In further examples, the clinical decision support tool 309 can be installed on the memory device 116 of the monitoring device 104 located inside the patient environment PE.

As shown in FIG. 9, the clinical decision support tool 309 includes one or more types of analyzers that include software programs and algorithms for analyzing the data acquired from the diagnostic imagers 108a, 108b. In the example shown in FIG. 9, the clinical decision support tool 309 is shown as including a wound analyzer 320, a neuro-ophthalmic analyzer 322, a gait analyzer 324, and a tremor analyzer 326. In further examples, the clinical decision support tool 309 can include additional types of analyzers, or fewer analyzers than the ones shown in FIG. 9.

The wound analyzer 320 can be used by the clinical decision support tool 309 to measure size, color, and temperature of a wound that is imaged by the diagnostic imager 108a, 108b during a telehealth consultation with a remote clinician. The size of the wound can include dimensions such as length, width, and depth. The color and temperature of the wound can indicate whether it is infected or not. These measurements can be used by the wound analyzer 320 to provide a recommendation for treating the wound to the remote clinician operating the telehealth device 300 such as to prescribe an antibiotic medication to treat an infection.

The neuro-ophthalmic analyzer 322 can be used by the clinical decision support tool 309 to conduct neurological assessments during a telehealth consultation. In one example, the neuro-ophthalmic analyzer 322 can be used by the clinical decision support tool 309 to conduct a stroke assessment during a telehealth consultation. This allows the telehealth device 300 provide telestroke services to local and regional healthcare facilities that do not have a neurologist onsite.

As an example, the neuro-ophthalmic analyzer 322 can perform a pupils equal, round, and reactive to light and accommodation (PERRLA) test on the patient during the telehealth consultation with the remote clinician. The PERRLA test is typically not performed during telehealth consultations because it is difficult for a remote clinician to view small changes in the pupils of a patient in response to changes in light intensity. However, the high resolution provided by the lens 140 and the image sensor 144, as well as the adjustable illumination provided by the illumination unit 146, allow the clinical decision support tool 309 to perform the PERRLA test by analyzing the reaction of the patient's pupils to changes in light intensity. Further, the neuro-ophthalmic analyzer 322 can automatically perform the PERRLA test during the telehealth consultation to remove human subjectivity and error from the analysis.

The gait analyzer 324 can be used by the clinical decision support tool 309 to detect gait disturbance, postural abnormalities, and other orthopedic conditions that can indicate that the patient is at risk of experiencing a patient fall which can cause serious health consequences. The gait analyzer 324 can include motion algorithms that can be used to measure patient lean. In some further examples, the gait analyzer 324 can compare a post-surgery image or video of the patient with a pre-surgical image or video of the patient to determine whether the patient has experienced deteriorated mobility. In some examples, video images of the patient's gait are recorded by panning the diagnostic imager 108a left and right about the first axis A-A and tilting the diagnostic imager 108a up and down about the second axis B-B (see FIG. 6) to follow the patient while the patient walks and moves around the patient environment PE.

The tremor analyzer 326 can be used by the clinical decision support tool 309 to detect whether the patient has a tremor, or whether a previously detected tremor of the patient has advanced due to Parkinson's disease, or has improved due to prescribed medications, treatments, and therapies. When the tremor analyzer 326 detects a tremor for the first time, or that a previously detected tremor has worsened, the clinical decision support tool 309 can recommend additional diagnostic testing and/or in-person assessment for follow-up.

FIG. 10 schematically illustrates an example of the robotic arm 106. FIG. 11 is an isometric view of the robotic arm 106. The robotic arm 106 can be used in combination with the monitoring device 104 and the diagnostic imager 108 to increase the ability for a remote clinician to provide healthcare inside the patient environment PE, especially when there is no local caregiver present inside the patient environment PE to provide assistance.

The robotic arm 106 is configured to function as an assistant for the remote clinician during a telehealth consultation. For example, the robotic arm 106 can be controlled by the remote clinician using the user interface 312 on the telehealth device 300 to generate control commands, and the communications interface 310 can send the control commands to the robotic arm 106 through the communications network 110 (see FIG. 1). The user interface 312 of the telehealth device 300 can include a joystick, a trackball mouse, or other type of input device for the remote clinician to generate the control commands for controlling the robotic arm 106 to perform medical procedures. As an illustrative example, the remote clinician can control the robotic arm 106 to perform medical procedures such as dressing changes, suture removal, staple removal, drain catchment replacement or drain removal, and other types of procedures.

Referring now to FIGS. 10 and 11, the robotic arm 106 includes a mount 160 that operates to fix the robotic arm 106 to a surface. In one example, the mount 160 fixes the robotic arm 106 to the monitoring device 104. In further examples, the mount 134 can fix the robotic arm 106 to a fixture such as a wall or ceiling in the patient environment PE, or to a piece of furniture or another device located inside the patient environment PE.

The robotic arm 106 includes a communications interface 172 that receives control commands from an external device such as the telehealth device 300 or the monitoring device 104 for execution by the robotic arm 106. The communications interface 172 can provide wired or wireless communications with the monitoring device 104 via a connection to the communications interface 118 (see FIG. 3). In further examples, the communications interface 172 can provide wired or wireless communications with the telehealth device 300 via a connection to the communications network 110. The communications interface 172 can include both wired interfaces (e.g., USB ports, etc.) and wireless interfaces (e.g., Bluetooth, etc.).

As further shown in FIGS. 10 and 11, the robotic arm 106 includes one or more arm joints 162 that pivotally and/or rotatably connect one or more arm links 164 to allow the robotic arm 106 to move in different directions when performing a medical procedure. In some examples, the robotic arm 106 is a 6-axis robotic arm having flexible movement. In some examples, the arm joints 162 provide about 180 to 360 degrees of motion for the arm links 164.

The robotic arm 106 includes end effectors 166 which are tools that attach to the distal end of the robotic arm 106. An end effector 166 allows the robotic arm 106 to perform tasks. In the example shown in FIG. 11, the end effector 166 is a gripper that can be used to grasp objects. Further examples of the end effectors 166 can include pincer type tools for removing surgical sutures and staples, suction tools, prodding tools, and other types of tools that can be used to perform various types of medical procedures. In some examples, the end effector 166 is replaceable such that one type of end effector (e.g., a gripper) can be replaced with another type of end effector (e.g., a suction tool) to perform different tasks on the patient.

In one example, an end effector 166 can be used to perform palpation on the patient during a telehealth consultation. For example, the end effector 166 can include a prodding tool that can be used to touch and push on a surface near a wound to observe blood perfusion. In some instances, the end effector 166 performs palpation by pushing down on a skin surface, and the diagnostic imager 108 can record changes in skin color to measure blood perfusion.

The robotic arm 106 further includes one or more electric motors 170 that operate to power the one or more arm joints 162 to pivot and rotate. The one or more electric motors 170 can have linear and rotary actuators powered by electric, hydraulic, or pneumatic systems. As the actuators move, they push and rotate the one or more arm links 164 into motion. Also, the one or more electric motors 170 power the end effector 166 to actuate in performance of a task.

The robotic arm 106 further includes one or more sensors 174, which detect and/or measure one or more parameters to trigger a corresponding reaction to them. The one or more sensors 174 are included for safety and control purposes. For example, the one or more sensors 174 can include safety sensors that are used to detect obstacles to prevent collisions. For example, a safety sensor can detect an obstacle, send a signal to a controller 168 which in turn slows or stops the robotic arm 106 to avoid a collision. Other parameters that the one or more sensors 174 can detect and/or measure include position, velocity, temperature, and torque.

In some examples, the robotic arm 106 can include the diagnostic imager 108 described above. In such examples, the diagnostic imager 108 can be positioned at the distal end of the robotic arm where the end effector 166 is attached. When the diagnostic imager 108 is included on the robotic arm 106, the diagnostic imager 108 does not need to be held or manipulated by the patient for imaging an area of interest, which can be especially advantageous when the patient does not have appropriate dexterity or familiarity with using the diagnostic imager 108. Instead, pitch, jaw, angle, and focus control can be adjusted by the robotic arm 106 to properly position and orientate the diagnostic imager 108 to capture well illuminated and high-resolution images for display on the display device 314 of the telehealth device 300.

The robotic arm 106 includes a controller 168, which is an example of a computing device. The controller 168 is programmed with software that enables the controller 168 to receive, interpret and execute control commands for controlling the operation of the robotic arm 106. For example, the controller 168 can instruct the electric motor 170 to pivot or rotate the one or more arm joints 162 based on control commands received from the telehealth device 300 or the monitoring device 104, and feedback received from the one or more sensors 174.

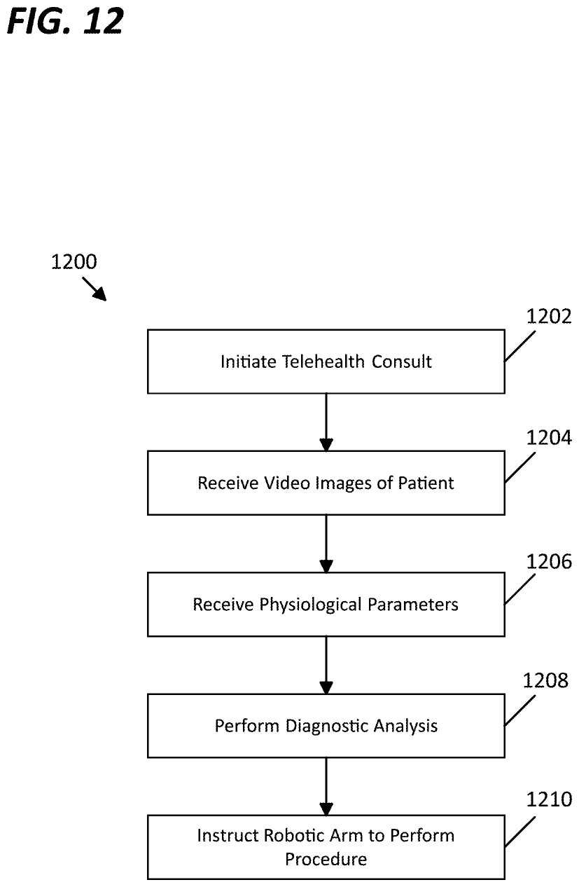

FIG. 12 schematically illustrates an example of a method 1200 of conducting a telehealth consultation with a patient in the patient environment PE. In some instances, the method 1200 can be performed by the telehealth device 300 in communication with the devices in the patient environment PE via the communications network 110 (see FIG. 1).

The method 1200 includes an operation 1202 of initiating a telehealth consultation. In some examples, the telehealth consultation is initiated on the telehealth device 300 following verification of the patient in accordance with the steps of the method 400 (see FIG. 4).

Next, the method 1200 includes an operation 1204 of receiving video images of the patient in the patient environment PE. In some examples, the video images received in operation 1204 are received from the camera 102 under the second modality which captures video data of high resolution and quality to aid assessment of the patient. In further examples, the video images received in operation 1204 are received from the diagnostic imager 108a shown in FIGS. 5 and 6, which can pan, tilt, and zoom for capturing well illuminated and high-resolution images of the patient. In further examples, the video images received in operation 1204 are received from the diagnostic imager 108b shown in FIGS. 7 and 8, which is a handheld portable imager that can be positioned and orientated to image hard to reach areas on the patient's body.

Next, the method 1200 includes an operation 1206 of receiving one or more physiological parameters of the patient in the patient environment PE. The one or more physiological parameters can be received from the physiological sensors 126 that are connected to the monitoring device 104. As an illustrative example, operation 1206 can include receiving at least one of blood oxygen saturation (SpO2), non-invasive blood pressure (systolic and diastolic), respiration rate, pulse rate, temperature, electrocardiogram (ECG), and heart rate variability. In some examples, operation 1206 can include receiving temperature readings from the temperature sensor 150 that is mounted on the diagnostic imager 108a, 108b.

Next, the method 1200 includes an operation 1208 of performing a diagnostic analysis on the patient in the patient environment PE. The diagnostic analysis can be performed by the clinical decision support tool 309. For example, the diagnostic analysis in operation 1208 can include at least one of: measuring size, color, and/or temperature of a wound imaged by the diagnostic imager 108a, 108b during the telehealth consultation (performed by the wound analyzer 320); conducting neurological assessments (e.g., stroke assessment, pupils equal, round, and reactive to light and accommodation (PERRLA) test, etc.) during the telehealth consultation (performed by the neuro-ophthalmic analyzer 322); detecting gait disturbance, postural abnormalities, and other orthopedic conditions that can indicate risk of patient fall (performed by the gait analyzer 324); and detecting whether the patient has a tremor, or whether a previously detected tremor has improved or deteriorated (performed by the tremor analyzer 326).

Next, the method 1200 includes an operation 1210 of instructing the robotic arm 106 to perform a procedure on the patient in the patient environment PE. Operation 1210 can be based on the diagnostic analysis performed in operation 1208. For example, when a diagnostic analysis indicates that a wound has properly healed, operation 1210 can include instructing the robotic arm 106 to remove surgical sutures and/or staples from the wound. As a further example, when the diagnostic analysis indicates a potential for infection, operation 1210 can include instructing the robotic arm 106 to perform palpation such as to measure blood perfusion.

The various embodiments described above are provided by way of illustration only and should not be construed to be limiting in any way. Various modifications can be made to the embodiments described above without departing from the true spirit and scope of the disclosure.

What is claimed is:

1. A method of remote monitoring a patient, the method comprising:
monitoring the patient under a first modality, wherein the first modality blocks identification of the patient;
determining whether an event is detected by the first modality; and
when an event is detected, monitoring the patient under a second modality, wherein the second modality is different from the first modality and includes capturing images of the patient.

2. The method of claim 1, wherein the first modality includes capturing light detection and ranging (lidar) data to detect a location of the patient inside a patient environment.

3. The method of claim 1, wherein the first modality includes transmitting millimeter waves to detect a location of the patient inside a patient environment.

4. The method of claim 1, wherein the first modality includes capturing the images under a first spectrum of light that obfuscates the patient, and the second modality includes capturing the images under a second spectrum of light that does not obfuscate the patient.

5. The method of claim 1, further comprising:
transferring a transmission of the images captured under the second modality to a device operated by a clinician authorized to view protected health information.

6. The method of claim 1, further comprising:
receiving a request from the patient to start a telehealth consultation;
obtaining a live image of the patient;
obtaining a stored image of the patient;
comparing the live image of the patient with the stored image of the patient; and
when the live image of the patient matches the stored image of the patient, initiating the telehealth consultation with a clinician remotely located with respect to the patient.

7. The method of claim 6, further comprising:
when the live image of the patient does not match the stored image of the patient, terminating the telehealth consultation.

8. The method of claim 6, wherein comparing the live image of the patient with the stored image of the patient includes using facial recognition technology.

9. The method of claim 6, wherein the stored image of the patient is obtained from an electronic medical record of the patient.

10. A method of conducting a telehealth consultation, the method comprising:
receiving images of a patient;
receiving one or more physiological parameters of the patient;
performing a diagnostic analysis on the patient based on at least one of the images and the one or more physiological parameters; and
instructing a robotic arm to perform a procedure based on the diagnostic analysis.

11. The method of claim 10, wherein the diagnostic analysis includes an analysis of a wound that uses the images to measure a size and a color of the wound, and the one or more physiological parameters include a contactless temperature reading of the wound.

12. The method of claim 11, wherein when the analysis of the wound indicates that the wound has properly healed, instructing the robotic arm to remove surgical sutures from the wound.

13. The method of claim 11, wherein when the analysis of the wound indicates a likelihood of infection, instructing the robotic arm to perform palpation for measuring blood perfusion.

14. The method of claim 10, wherein the diagnostic analysis includes conducting a neurological assessment during the telehealth consultation.

15. The method of claim 14, wherein the neurological assessment includes a pupils equal, round, and reactive to light and accommodation (PERRLA) test.

16. A telehealth device, comprising:
at least one processing device; and
a memory device storing instructions which, when executed by the at least one processing device, cause the telehealth device to:
control operation of a diagnostic imager for capturing images of a patient during a telehealth consultation;
analyze the images of the patient to determine a disease state; and
provide a clinical recommendation based on the disease state.

17. The telehealth device of claim 16, wherein analyze the images of the patient to determine the disease state includes measuring a size, a color, and a temperature of a wound.

18. The telehealth device of claim 16, wherein analyze the images of the patient to determine the disease state includes detecting gait disturbance, postural abnormalities, and orthopedic conditions indicative of a fall risk.

19. The telehealth device of claim 16, wherein analyze the images of the patient to determine the disease state includes detecting a tremor.

20. The telehealth device of claim 16, wherein control operation of the diagnostic imager includes controlling the diagnostic imager to pan, tilt, and zoom.
